(19) United States
(12) Patent Application Publication
(10) Pub. No.: US 2019/0167568 A1
(43) Pub. Date: Jun. 6, 2019

(54) Transdermal Drug Delivery System and Method for Using Same

(71) Applicants: SENJU USA, INC., Woodland Hills, CA (US); NICHIBAN CO., LTD., Tokyo (JP)

(72) Inventors: Takahiro OGAWA, Woodland Hills, CA (US); Akiharu ISOWAKI, Woodland Hills, CA (US); Tetsuo KIDA, Woodland Hills, CA (US); Koji KAWAHARA, Tokyo (JP); Emiko TESHIMA, Tokyo (JP); Teppei OSAKO, Tokyo (JP)

(73) Assignees: SENJU USA, INC., Woodland Hills, CA (US); NICHIBAN CO., LTD., Tokyo (JP)

(21) Appl. No.: 16/313,739

(22) PCT Filed: Jun. 29, 2017
(86) PCT No.: PCT/JP2017/024059

§ 371 Date: Dec. 27, 2018

Related U.S. Application Data
(60) Provisional application No. 62/355,974, filed on Jun. 29, 2016.

(51) Int. Cl. A61K 9/00; A61K 9/70; A61K 31/573

(52) U.S. Cl. CPC A61K 9/0014; A61K 9/7061; A61K 31/58; A61K 9/0048; A61K 9/7069; A61K 31/573

(57) ABSTRACT

[PROBLEMS TO BE SOLVED]

To provide a method of advantageously improving a transdermal permeation amount of a steroid drug to diseased portions when treating ophthalmic diseases such as chalazion, blepharitis, allergic conjunctivitis, vernal keratoconjunctivitis, and meibomian gland dysfunction.

[SOLUTION]

To provide the transdermal drug delivery system for administering a drug via the eyelid skins that have been treated with a microneedle array, featured in that the drug is a water-soluble steroid, and the transdermal drug delivery system is a water-containing base adhesive skin patch.

### TECHNICAL FIELD

The present invention relates to a transdermal drug delivery system, and a method for treating ophthalmic diseases by applying the transdermal drug delivery system to the eyelid skin (and its surrounding area) of a patient, the ophthalmic diseases being definable as chalazion, blepharitis, allergic conjunctivitis, vernal keratoconjunctivitis or meibomian gland dysfunction, and the like.

### BACKGROUND ART

Ophthalmic diseases (may be called eye or ophthalmological diseases) such as chalazion, blepharitis, allergic conjunctivitis, vernal keratoconjunctivitis, meibomian gland dysfunction, and the like are recognized due to the result of inflammation in general. Steroid drugs are used for treatment, and specifically the steroid drugs are to be provided in the form of an ophthalmic steroid ointment as an anti-inflammatory agent.

Conventionally, the long-term use of the ophthalmic steroid ointment has been problematic since it may cause serious side-effects such as increased intraocular pressure, cataract, corneal epithelial disorder and delayed wound healing, corticosteroid uveitis, mydriasis and ptosis, infectious diseases as well as transient ocular discomfort or steroid-induced calcium deposits (for example, J Am Acad Dermatol 2006; 54:1-15, Surv Ophthalmol 1979; 24:57-88., Br J Dermatol 1976; 95:207-8., Arch Dermatol 1976; 112:1326, Arch Dermatol 1978; 114: 953-4., Ophthalmology 1997; 104: 2112-2116).

Accordingly, effective and safe treatment for eye diseases such as chalazion, blepharitis, allergic conjunctivitis, vernal keratoconjunctivitis, meibomian gland dysfunction is widely demanded.

Patent Document 1 discloses a transdermal absorption-type preparation for treating ophthalmic diseases, the preparation being structured in such a way that a plaster layer containing an ophthalmic disease agent is provided on a backing film. Further, Patent Document 1 discloses a steroid patch having better effectiveness and safety than ophthalmic ointments, and a method for treating ophthalmic diseases.

Patent Document 2 discloses technical arts in which to administer drugs with a simple and compact transdermal drug administration device, in which medicine-containing hydrogel is placed on the skin surface after the microneedle array treatment has perforated the skin with a needle, the length of which is several hundred microns. Further, Patent Document 2 discloses that an amount of drug permeation is significantly improved by controlling the shape retention of the drug-containing hydrogel to be within a predetermined range.

### PRIOR ART DOCUMENT

### Patent Document

[Patent Document 1] Japanese National Publication No. 2014-519955 (SENJU USA)
[Patent Document 2] Japanese Patent No. 5767094 (Nichiban)

### Non-Patent Document

[Non-Patent Document 1] Journal of American Academy of Dermatology 2006; 54: 1-15
[Non-Patent Document 2] Survey of Ophthalmology 1979; 24: 57-88.
[Non-Patent Document 3] British Journal of Dermatology 1976; 95: 207-208
[Non-Patent Document 4] Archives of Dermatology 1976; 112:1326
[Non-Patent Document 5] Archives of Dermatology 1978; 114: 953-954
[Non-Patent Document 6] Ophthalmology 1997; 104: 2112-2116

### SUMMARY OF THE INVENTION

### Problems to be Solved by the Invention

The patent document 2 of the above does not disclose effective treatment of ophthalmic diseases such as chalazion, blepharitis, allergic conjunctivitis, vernal keratoconjunctivitis, meibomian gland dysfunction.

The present invention concerns the task of providing methods for advantageously improving a transdermal permeation amount of steroid drugs to diseased portions in the treatment of ophthalmic diseases such as chalazion, blepharitis, allergic conjunctivitis, vernal keratoconjunctivitis, and meibomian gland dysfunction.

### Means to Solve the Problems

The present inventors have found the following and have achieved this invention based thereon. Specifically, a water-containing adhesive skin patch with a water-soluble steroid drug is placed on the eyelid skin surface after microneedle perforation has been performed. This will notably improve an amount of transdermal permeation of drugs to ophthalmic diseased portions such as chalazion, blepharitis, allergic conjunctivitis, vernal keratoconjunctivitis, meibomian gland dysfunction. Accordingly, in the present invention, the sufficient amount of the drugs can be administered to the diseased portions in a short period of time.

That is, the present invention relates to a transdermal drug delivery system for administering a drug for treating ophthalmic diseases via the eyelid skin that has been treated by a microneedle array, wherein the drug is a water-soluble steroid, and the transdermal drug delivery system is a water-containing base adhesive skin patch.

According to the present invention, the following embodiments will be further provided.

[1] The transdermal drug delivery system is a water-containing adhesive skin patch comprising either adhesive hydrogel, or non-adhesive hydrogel and an adhesive tape for fixation, and the hydrogel contains a water-soluble steroid as the drug.

[2] The transdermal drug delivery system is a water-containing base adhesive skin patch obtained by which a water-containing base adhesive layer is arranged on a backing film, and the water-containing base adhesive layer contains a water-soluble steroid as the drug.

[3] The drug of the transdermal drug delivery system is at least one of the water-soluble steroids selectable from a range of −5 to 0 in octanol/water distribution coefficient (log D).

[4] The drug of the transdermal drug delivery system is at least one of the water-soluble steroids selectable from the group consisting of dexamethasone sodium phosphate, dexamethasone metasulfobenzoate sodium, hydrocortisone sodium phosphate, hydrocortisone sodium succinate, prednisolone sodium phosphate, prednisolone sodium succinate, methylprednisolone sodium succinate, and betamethasone sodium phosphate.

[5] The water-containing base adhesive layer or hydrogel of the transdermal drug delivery system contains polyvinyl alcohol.

[6] The water-containing base adhesive layer or hydrogel of the transdermal drug delivery system contains at least one kind selectable from the group consisting of polyacrylic acid and salt thereof.

[7] The ophthalmic disease of the transdermal drug delivery system is at least one disease selectable from the group consisting of chalazion, blepharitis, allergic conjunctivitis, vernal keratoconjunctivitis and meibomian gland dysfunction.

[8] It is a method of treating ophthalmic diseases, the method comprising: a process of perforating a microneedle on an eyelid skin surface of a patient by using a microneedle array; and a process of locally applying a transdermal drug delivery system to a portion of an eyelid skin on which perforation of the microneedle has been performed.

[9] It is a set of treating ophthalmic diseases, wherein the set includes a microneedle array for perforating an eyelid skin, a support base for eyelid skin, and a transdermal drug delivery system.

[10] The support base for eyelid skin in the set of treating ophthalmic diseases is either entropion forceps or a cornea protection plate (lid plate).

[11] It is a transdermal drug delivery system for administering a drug for treating ophthalmic diseases via an eyelid skin that has been treated by a microneedle array, wherein when the drug is a liposoluble steroid, the transdermal drug delivery system further comprises a water-soluble additive, the additive being produced by making the liposoluble steroid esterified to become water soluble, and the transdermal drug delivery system is a water-containing base adhesive skin patch.

### Effect of the Invention

According to the present invention, it is possible to significantly improve a transdermal permeation amount of steroid drugs to ophthalmic diseased portions such as chalazion, blepharitis, allergic conjunctiva disease, vernal keratoconjunctivitis, meibomian gland dysfunction. With this, the present invention enables the sufficient amount of the steroid drugs to quickly reach the diseased portions.

### BRIEF DESCRIPTION OF THE DRAWINGS

FIG. 1 is the diagram in Test Example 1, which shows the results of the skin permeability test using a water-containing base adhesive skin patch with a water-soluble steroid, with or without microneedle perforation.

FIG. 2 is the diagram in Test Example 1, which shows the results of the skin permeability test using a water-containing base adhesive skin patch with a liposoluble steroid, with or without microneedle perforation.

FIG. 3 is the diagram in Test Example 2, which shows the results of the skin permeability test using a water-containing base adhesive skin patch with a water-soluble steroid, with or without microneedle perforation.

FIG. 4 is the diagram in Test Example 2, which shows the results of the skin permeability test using a water-containing base adhesive skin patch with a liposoluble steroid, with or without microneedle perforation.

### EMBODIMENTS CARRYING OUT THE PRESENT INVENTION

[Transdermal Drug Delivery System]

The transdermal drug delivery system of the present invention is applied to a subject (for example, human, rabbit, dog, cat, cow, horse, monkey etc.) that has been infected with or that may be infected with ophthalmic diseases such as chalazion, blepharitis, allergic conjunctivitis, vernal keratoconjunctivitis or meibomian gland dysfunction. Specifically, it will be carried out in such a manner that a microneedle array with microneedles to be described later will perforate the subject's eyelid skin, and the transdermal drug delivery system as a water-containing adhesive skin patch is then applied to the portions on which the perforation has been done.

The eyelid skin mentioned above means a skin surface that includes the eyelid surface, more specifically it is the skin surface including front surfaces of the upper eyelid, the lower eyelid or the eyelids of both eyes, or each skin surface of these eyelids.

Accordingly, the transdermal drug delivery system according to the present invention preferably has a shape applicable along the skin surface of the upper eyelid, the lower eyelid or both eyelids. Specifically, those shapes will be, for example, a rectangular, elliptical, crescent, circular, horseshoe, ring, race-track shape and the like, allowing the shapes to go along the front surface of these eyelids.

The transdermal drug delivery system of the present invention may be of any size as long as it is attachable along the skin surface of the upper eyelid, the lower eyelid or both eyelids. The size thereof should be varied depending on which subject is to be used, but it may be, for example, 10 cm² or less. Preferably it may be 0.5 to 10 cm², more preferably 0.5 to 5 cm², particularly preferably 1 to 3 cm², and the most preferably about 1 cm².

The transdermal drug delivery system of the present invention is featured by being a water-containing adhesive skin patch, and it will be (1) the water-containing adhesive skin patch comprising either adhesive hydrogel, or non-adhesive hydrogel and an adhesive tape for fixation, or (2) the water-containing adhesive skin patch comprising a water-containing adhesive layer provided on a backing film. These will be that: a drug (water-soluble steroid) containing layer does not have adhesiveness to an eyelid skin or does not have such adhesiveness up to the level enabling the drug-containing layer to attach to the eyelid skin for a certain period of time (embodiment of (1)), or the drug-containing layer has adhesiveness to the eyelid skin (embodiment of (2)).

<Water Soluble Steroid>

A water-soluble steroid used for the transdermal drug delivery of the present invention may be any of the pharmacologically acceptable water-soluble steroids, for example, dexamethasones such as dexamethasone sodium phosphate and dexamethasone metasulfobenzoate sodium; hydrocortisones such as hydrocortisone sodium phosphate and hydrocortisone sodium succinate; prednisolones such as prednisolone sodium phosphate or prednisolone sodium succinate; methylprednisolones such as methylprednisolone sodium succinate; and betamethasones such as betamethasone sodium phosphate. Among them, dexamethasone sodium phosphate may be selected as a preferable drug.

The amount and dosage of the drug (water-soluble steroid) may vary depending on a drug type, or symptoms, age and body weight of each of the subject.

The above drug is blended with hydrogel or a water-containing base adhesive layer. For example, a blended ratio per 100 parts by mass of the hydrogel or water-containing adhesive layer may be: 0.00005 to 35 parts by mass, preferably 0.0005 to 15 parts by mass, and more preferably 0.005 to 7 parts by mass.

The administration time of the drug is not particularly limited, but for example, after conducting the microneedle perforation, the water-containing adhesive skin patch comprising the adhesive hydrogel, the water-containing adhesive skin patch comprising the non-adhesive hydrogel and the adhesive tape for fixation, or the water-containing adhesive skin patch prepared by providing the water-containing adhesive layer on the backing film may be applied for about 12 to 24 hours per day for continuously about 2 weeks. The microneedle perforation may be carried out 1 to 10 times, preferably 1 to 5 times, more preferably 1 to 3 times, immediately before the adhesive skin patch is applied.

<Liposoluble Steroid>

In the transdermal drug delivery system of the present invention, a liposoluble steroid may be used as the drug instead of the water-soluble steroid. In this case, a water-soluble additive is usable together with the liposoluble steroid, the water-soluble additive being produced by making the liposoluble steroid esterified to become water soluble (hereinafter simply referred to as “water-soluble additive”). This will make the liposoluble steroid able to function as the same with the case that the “water-soluble steroid” is administered.

The liposoluble steroid for use in the transdermal drug delivery system of the present invention may be any of the pharmacologically acceptable liposoluble steroids. This may be, for example, cortisone; hydrocortisones such as hydrocortisone, hydrocortisone acetate, hydrocortisone butyrate, and hydrocortisone butyrate propionate; prednisone; prednisolones such as prednisolone, prednisolone acetate, prednisolone valerate acetate, and methylprednisolone; triamcinolone; paramethasone; dexamethasones such as dexamethasone, dexamethasone acetate, dexamethasone valerate, and dexamethasone propionate; betamethasones such as betamethasone, betamethasone valerate, betamethasone butyrate propionate, and betamethasone dipropionate; clobetasones such as clobetasone, and clobetasone butyrate; triamcinolone acetonide; fluocinolone acetonide; alclometasones such as alclometasone and alclometasone dipropionate; beclomethasones such as beclomethasone and beclomethasone dipropionate; deprodones such as deprodone and deprodone propionate; mometasones such as mometasone and mometasone furoate; amcinonide; halcinonide; fluocinonide; diflucortolones such as diflucortolone and diflucortolone valerate; budesonide; difluprednate; diflorasones such as diflorasone and diflorasone diacetate; clobetasol; clobetasols such as clobetasol propionate; halobetasols such as halobetasol, and halobetasol propionate; fluorometholones such as fluorometholone and fluorometholone acetate; loteprednols such as loteprednol and loteprednol etabonate; male hormones such as androgen, testosterone and dihydrotestosterone; and female hormones such as estrogen, estradiol, and estriol. Among them, clobetasol and clobetasols such as clobetasol propionate may be selected as preferable drugs.

<Water-Soluble Additive>

Examples of the water-soluble additives to be used with the liposoluble steroid may be phosphoric acid, sulfuric acid, carbonic acid, nitric acid, 2-sulfobenzoic acid, 3-sulfobenzoic acid, 4-sulfobenzoic acid, oxalic acid, malonic acid, succinic acid, glutaric acid, adipic acid, pimelic acid, suberic acid, azelaic acid, sebacic acid, phthalic acid, isophthalic acid, terephthalic acid, poly (ethylene glycol) bis (carboxymethyl) ether, methoxypolyethylene glycol acetate, O-methyl-O′-succinic acid polyethylene glycol and the like.

The blending proportion of the water-soluble additive varies depending on the type of liposoluble steroid used, but it is normally set to 1 to 50 equivalent %, preferably 1 to 20 equivalent %, more preferably 1 to 5 equivalent % with respect to the liposoluble steroid.

When using the liposoluble steroid, it can be mixed with the water-soluble additive to produce the water-containing base adhesive skin patch as described later.

In the present invention, the distribution coefficient (octanol/water distribution coefficient) may be adopted as the index of “water soluble” and “liposoluble” in the “water soluble steroid” and the “liposoluble steroid.” The distribution coefficient can be expressed in log D in consideration of the influence of pH, and water solubility is indicated by a minus value while liposolubility is indicated by a plus value.

The range of the distribution coefficient (log D) of compounds to be used is not particularly limited, but from the viewpoint of skin permeability, it is preferable to use the compounds ranging from −5 to 0 at pH 8.

For example, as examples of the compounds of the above water soluble steroid under the distribution coefficient (log D) at pH 8, dexamethasone sodium phosphate: −5, dexamethasone metasulfobenzoate sodium: −2, prednisolone sodium phosphate: 0, betamethasone sodium phosphate: −5 may be selected.

The distribution coefficient can be obtained according to the method of JIS 27260-107 or JIS 26260-117.

<(1) A Water-Containing Adhesive Skin Patch Comprising Adhesive Hydrogel, or a Water-Containing Adhesive Skin Patch Comprising Non-Adhesive Hydrogel and an Adhesive Tape for Fixation>

In the present embodiment, adhesive hydrogel or non-adhesive hydrogel (hereinafter may collectively referred to as “hydrogel”) includes a water-soluble steroid as the drug mentioned above, a water-soluble polymer as a base material, water, and optionally other commonly used components.

The term “adhesiveness” in hydrogel of the present specification means that it has a tack thereby containing certain adhesion to human skin to such an extent that a patch is not easily shifted from diseased portions. As the rough indication of the tack here, for example, though not limited thereto, 4 or more tacks may be provided in a rolling tack (JIS Z0237).

The water-soluble polymer of the above is a general term for polymer compounds which are soluble in water, and as long as it does not affect others, a hydrogel or one commonly used in the technical fields of a water-containing adhesive skin patch to be described later may be used. For example, polyacrylic acid salt such as polyacrylic acid and sodium polyacrylate; a partially neutralized material of polyacrylic acid such as acrylic acid-sodium acrylate copolymer; polyvinyl alcohol; polyvinyl-pyrrolidone; polyacrylamide; cellulose derivatives such as methyl cellulose, ethyl cellulose, hydroxyethyl cellulose, carboxymethyl cellulose, sodium carboxymethyl cellulose; and natural polymers such as gum arabic, gum tragacanth, gellan gum, agar, starch, alginic acid and metal salt thereof, gelatin and casein, and the like may be mentioned. These water-soluble polymers may be used alone or in combination of two or more, or may be frozen or irradiated with radiation. Among them, polyvinyl alcohol, or polyacrylic acid and salt thereof are preferably used as the water-soluble polymer.

The blending amount of the water-soluble polymer is, relative to the total mass of hydrogel, for example, 1% by mass to 50% by mass, preferably 5% by mass to 40% by mass, more specifically 3% by mass to 40% by mass, and still more preferably 5% by mass to 30% by mass. When the blending amount is less than the above-mentioned numerical range, the shape retention deteriorates. On the other hand, when it exceeds the above numerical range, the water-soluble polymer does not dissolve uniformly in the formation material of hydrogel described later, and the viscosity thereof increases. This will deteriorate operability in production so as to produce non-uniform hydrogel, which is not desirable.

The blending quantity of water contained in hydrogel may be, for example, 50 to 95% by mass with respect to the total mass of hydrogel.

When the water content is set to low, solubility of components such as drugs in the forming material of hydrogel becomes lowered thereby precipitating crystals. Further, when the viscosity of the forming material is too high, operability in production may be lowered. Still further, skin irritation such as pain may occur when releasing the adhesive skin patch from skin after the patch has been applied on the skin. On the other hand, if the water content is too large, the shape retention of hydrogel may be deteriorated due to viscosity decrease of the forming material of hydrogel, and tackiness of hydrogel or the like may occur. Moreover, since water volatility is enhanced, quality for the adhesive skin patch may not be kept at the time of storage or administration.

Hydrogel of the above may further contain a crosslinking agent, alcohol and polyhydric alcohol, solvent, a skin absorption assistant, humectant, tackifier resin, surfactant, a pH regulator, a filler, antioxidant, an UV absorbent, and other commonly used components such as a preservative. In consideration of the shape retention of hydrogel itself, skin permeability of drug, irritation to skin, etc., the type and amount of the other commonly used components may be appropriately selected.

The type of the crosslinking agent is not particularly limited, but the examples thereof may include a polyvalent metal compound, a boric acid base compound, a polyfunctional epoxy compound, and the like. These crosslinking agents may be used alone or in combination of two or more.

As the polyvalent metal compound, aluminum compounds is preferable, and the examples thereof may include a dry aluminum hydroxide gel, aluminum hydroxide, aluminum chloride, aluminum sulfate, dihydroxyaluminum aminoacetate, kaolin, aluminum stearate, magnesium metasilicate aluminate, magnesium silicate aluminate, synthetic hydrotalcite, potassium aluminum sulfate (potassium alum), ammonium alumina sulfate (ammonium alum), synthetic aluminum silicate, aluminum metasilicate, basic aluminum acetate, activated alumina and the like. Further, magnesium metasilicate, magnesium silicate and the like may be also included.

Examples of the boric acid compound include boric acid, ammonium borate, calcium borate, sodium metaborate, sodium tetraborate and the like.

Examples of the polyfunctional epoxy compound include sorbitol polyglycidyl ether, polyglycerol polyglycidyl ether, diglycerol polyglycidyl ether, glycerol polyglycidyl ether, ethylene glycol diglycidyl ether, polyethylene glycol diglycidyl ether, polypropylene glycol diglycidyl ether and the like.

When blending the crosslinking agent, the blending amount varies depending on the type of the crosslinking agent, but, based on the total mass of hydrogel, it is preferably, for example, 0.01% by mass to 30% by mass, specifically, 0.1% by mass to 30% by mass, more preferably 0.1% by mass to 10% by mass. If the blending amount of the crosslinking agent is less than the above numerical range, the effect of adding the crosslinking agent may not be sufficiently obtained, that is, the crosslinking of the water-soluble polymer does not sufficiently proceed, and viscosity of the formation material of hydrogel becomes too low, so that its formability may be deteriorated. On the other hand, when the crosslinking agent is added beyond the above-mentioned numerical range, the crosslinking of the water-soluble polymer proceeds excessively, causing rapid increase of viscosity in the formation material of hydrogel. Accordingly, this may cause the lack of homogeneity in the formation material of hydrogel, deteriorate operability of production, and increase skin irritation in the hydrogel that has been formed.

The alcohol and the polyhydric alcohol may have functions of, for example solvent, a skin absorption assistant, humectant and the like, and the examples thereof include ethanol; glycerin, ethylene glycol, diethylene glycol, triethylene glycol, polyethylene glycol, propylene glycol, polypropylene glycol and the like; 1, 3-propanediol, 1,3-butanediol, 1,4-butanediol, 1,2,6-hexanetriol and the like; D-sorbitol, xylitol, mannitol, erythritol and the like.

The blending amount of these alcohols is not particularly limited, but it can be set, for example, 0.1% by mass to 60% by mass with respect to the total mass of hydrogel.

The examples of the skin absorption assistant further include fatty acids and esters thereof such as lactic acid, oleic acid, linoleic acid, myristic acid and the like; and animal and vegetable oils as well as terpene compounds such as mint oil, I-menthol, dl-camphor and N-methyl-2-pyrrolidone, and the like.

Further, as the surfactant with some required quantities, sorbitan monooleate, polyoxyethylene sorbitan monooleate, and the like are used; as the pH regulator, tartaric acid, citric acid, and the like are used; and as the filler, bentonite, kaolin, talc, titanium white, and the like are used.

The blending amount of these is not particularly limited, but it may be, for example, in the range of, 0.1% by mass to 15% by mass, preferably 0.5% by mass to 10% by mass with respect to the total mass of hydrogel.

The method for producing hydrogel of the above is not particularly limited. As one of the examples, the water-soluble steroid may be dissolved in a suitable dissolving agent (water, solvent, etc.) if necessary; a water-soluble polymer, water, various components are added thereinto as base materials; it is heated if necessary and mixed evenly; and then it is left as rest in order to obtain the hydrogel.

A transdermal permeation amount of drugs contained in hydrogel can be improved regardless of types of base materials (polymer) as constituent materials of gel, by setting a stress relaxation rate of drug-containing hydrogel after 5 minutes to 35 to 80%, preferably. The “stress relaxation rate after 5 minutes” is a value obtained by the measurement method disclosed in the specification of Japanese Patent No. 5767094 (the contents of which are referred to and introduced in this specification). Specifically, a probe is applied to gel with a certain load, and values will be then calculated as the percentage of variation in load after 5 minutes with respect to an initial load.

Although the thickness of the hydrogel is appropriately set depending on the required drug concentration, sustainability and the like, it will be, for example, about 1 to 5000 μm.

In order to improve operability of hydrogel, a sheet-shaped backing film having anchoring properties may be disposed on one surface of the hydrogel, and in the case of non-adhesive hydrogel described later, it is possible to cover the backing film with the adhesive tape for fixation. As the backing film that is applicable in this case, the backing film to be mentioned in the water-containing adhesive skin patch of (2) below will be suitable.

In the case of the non-adhesive hydrogel of the present embodiment, that is, when hydrogel itself does not have adhesive properties, or hydrogel does not have sufficient adhesiveness to eyelid skins for a certain period of time, it is possible to adopt the adhesive skin patch on which the adhesive tape for fixation is covered when applied to the eyelid skins. This adhesive tape for fixation is covered over the non-adhesive hydrogel, or covered over the backing film on which the non-adhesive hydrogel is laminated. By fixing the non-adhesive hydrogel with the adhesive tape for fixation, moderate pressure is given to skin whereby the non-adhesive hydrogel can enter perforation that have been perforated by a microneedle described later. Accordingly, drugs contained in the non-adhesive hydrogel (water-soluble steroid) can be effectively administered.

Note that, even when using the adhesive hydrogel, it may be fixed with the adhesive tape for fixation, if necessary.

The adhesive tape for fixation may be, for example, composed of a tape base material and an adhesive layer.

The material of the tape base material is not particularly limited but may be selected from polyethylene; polypropylene; ethylene-vinyl acetate copolymer; ethylene-vinyl alcohol copolymer; vinyl chloride; polyurethane; polyester such as polyethylene terephthalate, polybutylene terephthalate, polyethylene naphthalate; polyamide such as nylon; polyacrylonitrile; cellulose or the derivative thereof; metal foil such as aluminum. It will be preferable to make the tape base material being film or fabric (woven fabric, nonwoven fabric, knitted fabric) by combining one or two more kinds of the material selected from the above. Among the above, polyurethane will be preferably used. The thickness of the tape base material is preferably 1 to 200 μm, more preferably 5 to 150 μm, yet further preferably 10 to 100 μm. If the thickness of the tape base material is too thin, it may cause shape impairment such as tear due to strength decrease and make it difficult to adhere skin patches to skin. On the other hand, if the thickness of the adhesive tape for fixation is too thick, it may cause usability deterioration or lifting of the skin patch in use, causing skin discomfort.

For the adhesive layer of the adhesive tape for fixation, various kinds of adhesives such as natural rubber type, synthetic rubber type, acrylic type, silicone type, polyvinyl alcohol type, polyamide type and the like may be used. In this regard, by using an acrylic-typed adhesive, it is possible to economically obtain the adhesive tape with relatively little stimulation to skin. The thickness of the adhesive layer is preferably 5 to 200 μm, and more preferably 5 to 50 μm. When the thickness of the adhesive layer is too thin, adhesive force to skin will decrease, causing the adhesive tape to be easily released. On the other hand, when the thickness of the adhesive layer is too thick, it may cause usability deterioration and a residue of adhesives, giving discomfort to a user.

For improving releasability or adhesiveness, the adhesive tape for fixture may be subjected to the process of secant lines, perforations, carrier sheets and the like. Further, for improving appearance or for preventing a film from being slipped off, it may be necessary to perform embossing or the like as appropriate.

<(2) Water-Containing Adhesive Skin Patch with a Water-Containing Adhesive Layer Provided on a Backing Film>

In this embodiment, the water-containing adhesive layer contains a water-soluble steroid as the drug mentioned above, a water-soluble polymer as a base material, and water.

Further, the water-containing base adhesive layer may properly contain other commonly used components, for example, a crosslinking agent, alcohol and polyhydric alcohol, solvent, a skin absorption assistant, humectant, tackifier resin, surfactant, a pH regulator, a filler, antioxidant, ultraviolet absorbent, antiseptic, and the like. For other commonly used components, each kind and blending amount thereof can be appropriately selected in consideration of shape retaining properties of the water-containing adhesive skin patch itself, adhesion to skin, skin permeability of drug, skin irritation, and the like.

As these water-soluble polymers or other commonly used components, the compounds described in hydrogel of the above (1) may be properly used.

The blending amount of each component in the water-containing base adhesive layer is not particularly limited, but with respect to the total mass of the water-containing adhesive layer, the blending amount of the water-soluble polymer may be the range of, for example, 1% by mass to 50% by mass, preferably 5% by mass to 40% by mass, and more preferably 5% by mass to 30% by mass. When the blending amount is less than the above numerical range, shape retention and adhesive strength are not exerted. On the other hand, when the blending amount exceeds the above numerical range, the water-soluble polymer does not dissolve uniformly in the formation material of the adhesive layer to be explained later. Further, this will expand viscosity thereby lowering production operability and forming uneven adhesive layers, which are not desirable.

The blending amount of water contained in the water-containing based adhesive layer may be, with respect to 100 parts by mass of the above water-soluble polymer, the range of, for example, 200 parts by mass to 2000 parts by mass, preferably 400 parts by mass to 1800 parts by mass, and more preferably 600 parts by mass to 1600 parts by mass.

When the water content is low, solubility of the components such as drugs in the formation material of the adhesive layer becomes lowered so as to precipitate crystals, and further viscosity of the material becomes too high, so that operability will be deteriorated when spreading the material onto the backing film. Accordingly, operability in production will decrease. Moreover, it is possible that adhesiveness becomes too hard, so that skin irritation may occur when releasing the adhesive skin patch from skin after the patch has been applied. On the other hand, when the water content is too large, shape retention of the adhesive layer may be deteriorated due to viscosity decrease of the formation material of the adhesive layer. In addition, tackiness on the adhesive layer may cause, and adhesive force may decrease. Still further, since water volatility will become high, it will be not assured that quality can be held during storage or administration.

When blending the crosslinking agent, the blending amount varies depending on the type of the crosslinking agent, but it can be 0.01% by mass to 10% by mass relative to the total mass of the water-containing adhesive layer. If the blending amount of the crosslinking agent is less than the above numerical range, positive effects through the addition of the crosslinking agent may not be sufficiently obtained. Thus, the crosslinking of the water soluble polymer does not proceed sufficiently, and viscosity of the formation material of the adhesive layer becomes too low, deteriorating formability. Moreover, it may be possible that adhesiveness is not sufficiently to be revealed. On the other hand, if the crosslinking agent is blended in an amount exceeding the above-mentioned numerical range, the crosslinking of the water-soluble polymer progresses excessively, causing a rapid increase in viscosity of the formation material of the adhesive layer. This may cause deterioration of operability in production, such as the lack of uniformity in the formation material of the adhesive layer or uneven spreading of the formation material of the adhesive layer onto the backing film. Moreover, adhesive force of the adhesive layer may become too hard, possibly causing high skin irritation.

In addition, when alcohols are blended, the blending amount thereof is not particularly limited, but it may be, for example, 0.1% by mass to 60% by mass relative to the total mass of the water-containing base adhesive layer.

Further, when a skin absorption assistant, surfactant, a pH regulator, a filler and the like are used, each blending amount of these with respect to the total mass of the water-containing adhesive layer may be in the range of, for example, 0.1% by mass to 15% by mass and preferably 0.5% by mass to 10% by mass.

The water-containing adhesive skin patch obtained by placing the water-containing adhesive layer on the backing film can be produced according to a general method of producing water-containing external patches. For example, the above water-soluble steroid is dissolved in a suitable dissolving agent (water, solvent, etc.) if necessary, and a water-soluble polymer, water and various component are added thereto as a base material and mixed so as to prepare the formation material of the adhesive layer. Then, the formation material of the adhesive layer is spread over a proper backing film to be described later in order to form the water-containing adhesive layer.

The thickness of the water-containing adhesive layer is not particularly limited, but it may be appropriately selected within the range of, for example, 10 to 300 μm in consideration of skin permeability of water-soluble steroids, adhesiveness to skin, and the like.

As the backing film for use in the water-containing adhesive skin patch provided with the water-containing adhesive layer on the backing film, it is not particularly limited if it is the backing film that is made of a material having flexibility to the extent that it can be brought into close contact with the skin surface including the front face of undulated eyelids. Note that those commonly used in the technical fields of patches may be used. For example, it is preferable to use materials in which: drugs do not easily exude from the water-containing adhesive layer; the backing film does not absorb drugs (water-soluble steroid) contained in the adhesive layer, or the drugs are not released from the back side of the backing film Note that the backing film of the above materials is also usable as a backing film for improving operability of hydrogel of the above (1).

Specific examples of the backing film include nonwoven fabrics, woven fabrics, knitted fabrics, films or sheets, porous materials, foams, paper, and composite materials obtained by laminating two or more of these materials.

Examples of the nonwoven fabrics may be fibrous materials including polyolefin resins such as polyethylene, polypropylene, and the like; polyester resins such as polyethylene terephthalate, polybutylene terephthalate, polyethylene naphthalate, and the like; rayon; polyamide; polyester ether; polyurethane; polyacrylic resin; polyvinyl alcohol; styrene-isoprene-styrene copolymer; styrene-ethylene-propylene-styrene copolymer, and the like.

Further, examples of the woven fabric and knitted fabric may be fibrous materials including cotton, rayon, polyacrylic resin, polyester resin and polyvinyl alcohol, and the like.

Examples of the film or the sheet include polyolefin resins such as polyethylene, polypropylene, and the like; polyacrylic resins such as polymethyl methacrylate, polyethyl methacrylate, and the like; polyester resins such as polyethylene terephthalate, polybutylene terephthalate, polyethylene naphthalate, and the like; cellophane; polycarbonate; polyvinyl alcohol; ethylene-vinyl alcohol copolymer; polyvinyl chloride; polystyrene; polyurethane; polyacrylonitrile; fluororesin; styrene-isoprene-styrene copolymer; styrene-butadiene rubber; polybutadiene; ethylene-vinyl acetate copolymer; polyamide; polysulfone, and the like. The materials are however not limited thereto.

Examples of the paper include impregnated paper; coated paper; high-quality paper; kraft paper; Japanese paper; glassine paper; synthetic paper, and the like.

Among them, in view of close adherability to eyelid skins, followability to movement of the eyelid skins and suppressionability to rash occurrences after a long-term attachment, polyester film, polyurethane film or polyolefin film will be preferable. Note that polyester films (especially polyethylene terephthalate films) will be particularly preferred.

The thickness of the backing film is not particularly limited, but it is usually in the range of 1 to 80 μm, preferably 2 to 70 μm, and more preferably 5 to 60 μm. When the thickness of the backing film is too small, the strength of the backing film becomes insufficient. Accordingly, the backing film may be torn when attaching to eyelids or when being released from the eyelids. This thus makes difficult to produce the backing film. On the other hand, if the thickness of the backing film is too large, this will make the thickness of the water-containing adhesive skin patch per se become large. Accordingly, the adhesive skin patch may not adhere tightly along the skin surface of the eyelids having fine irregularities such as epidermal depression, may become conspicuous in an attached condition, may increase discomfort to a user and may enlarge pain when the adhesive skin patch is released from skin. The thickness of the backing film is measured using a dial thickness gauge. Note that the same measuring method may apply when measuring the thickness of other layers in the adhesive skin patch in the treatment of ophthalmic diseases.

Further, the backing film preferably has flexibility to the extent that it can be brought into close contact with an eyelid skin and can follow movement of the eyelid skin. For example, the Young's modulus may have an elastic modulus of 0.01 to 0.5 GPa, preferably 0.03 to 0.48 GPa, more preferably 0.05 to 0.45 GPa. If the Young's modulus of the backing film is too small, there is a risk that the strength of the adhesive skin patch will become insufficient. Further, if the adhesive skin patch for treating ophthalmic diseases is applied to eyelid skins, it may be torn during application or when released from skin after certain periods of time. On the other hand, when the Young's modulus of the backing film is too large, there is a risk that adhesiveness of the adhesive skin patch to eyelid skins and followability to movement of the eyelid skins may become inferior. Accordingly, there is a possible risk that the adhesive skin patch may be released or float from skin immediately after the application, preventing the adhesive skin patch from a long-term application.

The backing film with the elastic moduli of the above numerical range is not particularly limited, but in many cases films and sheets of the various resins described above may be selected. The Young's moduli of these films or the like are measurable in accordance with ASTM-D-882, and it would be preferable that at least one direction but possibly in both directions of the Young's moduli in a MD direction (that is, extrusion direction at film formation) and a TD direction (perpendicular to the extrusion direction at film formation) of a film have the elastic moduli in the above numerical range.

<Release Film>

In the transdermal drug delivery system of the present invention, a release film may be provided on: a surface of hydrogel in embodiment (1) of the above; a surface of hydrogel opposite to the surface on which the backing film is placed if the backing film is arranged on one surface of hydrogel; and a surface of hydrogel opposite to the surface on which an adhesive tape for fixation is provided in the case that hydrogel is non-adhesive. Further, the release film may be provided for protecting the surface of the water-containing adhesive layer in embodiment (2) of the above. The release film (also referred to as a release liner, release paper, etc.) is to be released when the transdermal drug delivery system is used. This release film is to protect a layer that meets an eyelid skin until actually being used so as to prevent qualitative deterioration. For the release film, those conventionally used in the technical fields of transdermal absorption preparations or patch products (patch materials, adhesive skin patches) may be used, and examples thereof may be colorless or colored sheets including: plastic films such as polyester (polyethylene terephthalate, polybutylene terephthalate, polyethylene naphthalate, etc.), polypropylene (unstretched, stretched, etc.), polyethylene, polyurethane, ethylene-vinyl acetate copolymer, ethylene-vinyl alcohol copolymer, polyvinyl chloride, polystyrene, polyamide, polyacrylonitrile and the like; papers or synthetic papers such as high-quality paper, glassine paper, parchment paper, kraft paper, and the like; releasable processed papers made by which a release agent having release capacity such as silicone resin or fluorine resin coats the above-mentioned plastic film, paper or synthetic paper, synthetic fiber or the like; aluminum foil; laminated process paper made by which these films or sheets are variously laminated; laminate releasable process paper made by which a release agent coats the laminated process paper, and the like.

The thickness of the release film is not particularly limited but may be normally selected within the range of 10 μm to 1 mm, specifically, 20 to 500 μm, preferably 40 to 200 μm, more preferably 40 to 150 μm, further preferably 40 to 120 μm, and particularly preferably 50 to 100 μm. If the release film is too thin, it tends to cause shape breakage such as tear due to strength deterioration, occur problems in the production of the transdermal drug delivery system, or arise some difficulties to attach the water-containing adhesive skin patch to eyelid skins. On the other hand, if the release film is too thick, it may lead to poor cutting suitability of the release film in the production of the transdermal drug delivery system, to curling when the water-containing adhesive skin patch is attached, to increase of a raw-material cost, and the like.

In addition, the shape of the release film can be a square, a rectangle, a circle, etc., and it is possible to have a rounded corner if desired. The size of the release film in the embodiment (1) may be the same with or slightly larger than the size of the adhesive tape for fixation, and in the embodiment (2), it may be the size of the backing film in the water-containing base adhesive skin patch. The release film may be composed of one piece or divided pieces, and its break may be a straight line, a wavy line, or a perforated line. A part of the release films may be overlapped. Further, embossing treatment or the like may be performed in order to: print the kinds or usage of drugs; improve appearance or prevent the shift of a film; and easily take out the adhesive skin patch from a packaging material.

[Microneedle Array (MNA)]

The transdermal drug delivery system of the present invention is used for administering a drug via the eyelid skin that has been treated by an MNA, that is, in advance of the application of the transdermal drug delivery system of the present invention, the MNA treatment will be performed to the eyelid skin.

The specific method and mode of the MNA treatment is not particularly limited, and may be performed with, for examples, any tools that enable to temporarily reduce barrier functions of the eyelid skin by perforating the skin with a plurality of needles at the same time (See, for example, Wu, X. M. et al. (2006) J. Control Release, 118: 189-195 etc.).

There are no particular restrictions on constituent materials of the microneedle constituting the MNA used for the above MNA treatment, and examples thereof may include: a synthetic plastic microneedle with a base material of polycarbonate, polyurethane, polymethacrylate, ethylene-vinyl acetate copolymer, polytetrafluoroethylene, polyoxymethylene, polyester, nylon, polystyrene or polyolefin; an autolysis microneedle with a base material of polylactic acid, polycaprolactone, polyglycolic acid; or a microneedle made of silicon (compound), silicon dioxide, ceramic, metal (stainless steel, iron, aluminum, titanium, nickel, etc.). The shape and size of the microneedle are also not particularly limited, but normally it has a conical shape such as a circular-cone shape or a polygonal pyramidal shape (a triangular pyramidal shape, a quadrangular pyramidal shape or the like). When the microneedle has the triangular pyramidal shape, it may be set as that the area of the bottom surface thereof is about 0.1 to 0.5 mm², the height of the pyramidal shape thereof is about 0.2 to 0.5 mm, and the conical tip diameter is about 1 to 30 μm.

The size of the MNA is not particularly limited based on the area to which the transdermal drug delivery system of the present invention is applied, and it may be, for example, that the size of the MNA may be equal to, smaller than or larger than the area to which the transdermal drug delivery system is applied. However the area can preferably be the one equal to or smaller than the application area of the system.

Further, the set number (or the number) of the microneedles constituting the MNA may be properly set, but it may be the number of 1 to 500.

The transdermal drug delivery system of the present invention structured as discussed hereinabove further increases skin permeability of a steroid drug so as to enable a sufficient amount of the steroid drug to quickly reach diseased portions of the eyelid. Accordingly, since this will help to heal and cure inflammation of the diseased portions in a short period of time, users expect that the transdermal drug delivery system of the present invention will be effective from the perspective of: reducing the burden of taking a drug, suppressing the side-effects caused by prolonged use of the drug and improving administration compliance of the drug.

[Set of Treating for Ophthalmic Diseases]

The present invention will also deal with a set of treating ophthalmic diseases, including a microneedle array for perforating eyelid skins, a support base for eyelid skin, and a transdermal drug delivery system.

The transdermal drug delivery system and the microneedle array for perforating the eyelid skin in this set are applicable by the transdermal drug delivery system of the present invention and the microneedle array used for the microneedle array treatment in the transdermal drug delivery system of the present invention.

<Support Base for Eyelid Skin>

The eyelid skin rich in flexibility may not be sufficiently perforated only by using conventional microneedle array treatment. Accordingly, there have been some notable problems because the effects of improving drug permeation cannot be obtained. On the other hand, by providing certain stiffness to the skin to be perforated, sufficient perforation of the microneedle will become possible. As the method for giving certain stiffness to skin, the support base to support skin will be used.

Specifically, the support base for eyelid skin is a base for supporting an eyelid, the base being used by, for example, making it inserted into a gap between an eyelid skin and an eyeball when the eyelid skin is perforated with the microneedle array.

The support base for eyelid skin is inserted, for example, into a gap between the eyelid skin and the eyeball, that is, being inserted from a conjunctiva inside the eyelid, which is the side opposite to the skin surface to be perforated. During the microneedle array treatment, the support base is arranged in such a manner as to sandwich the eyelid skin between the microneedle array and the support base for eyelid skin

The above support base for eyelid is not particularly limited as long as it has the size and thickness that can be inserted between the eyelid skin and the eyeball, and has the above-mentioned functions. For example, entropion forceps or a cornea protection plate (can be also called lid plate or tapetum) may be used. The cornea protection plate (lid plate) means one of the medical instruments commonly used in ophthalmology. When using the entropion forceps, the pinching face (plate-like member) of the entropion forceps is inserted into a conjunctiva inside the eyelid opposite to the skin side to be perforated. Then, by using the frame (window portion) of the entropion forceps, an eyelid skin is pinched from the skin side to be perforated so as to stretch the skin to give tension. With this, the perforation treatment will be performed with the microneedle within the frame (window) of the entropion forceps. On the other hand, when using the cornea protection plate (lid plate), the contact surface of the cornea protection plate (lid plate) on the side of the eyelid is inserted into a conjunctiva inside the eyelid opposite to the skin side to be perforated. Then, the perforation treatment with the microneedle will be performed to the eyelid skin, which is supported by the cornea protection plate (lid plate). In addition, the microneedle array may have the functions (or parts) of the support base for eyelid. With the microneedle device having the function of the support base for eyelid, the microneedle device will enable not only to pinch an eyelid but also to perforate the eyelid.

### EMBODIMENT

Hereinafter, the present invention will be described in more detail based on embodiments. These examples of pharmaceutical preparation and embodiments are merely illustrative, and are not intended to limit the scope of the present invention. In this embodiment, “%” in the composition ratio of a mixture means “mass %.”

### Production Example: Production of Water-Containing Adhesive Skin Patch

A water-containing adhesive skin patch was produced according to the following procedure.

### Example 1: Water-Containing Base Adhesive Skin Patch with Water-Soluble Steroid

By using dexamethasone sodium phosphate (DSP) as a water-soluble steroid, DSP, polyvinyl alcohol (PVA, Kuraray Co., Ltd., polymerization degree: 1700) and water were mixed with the mass ratio of DSP/PVA/water=25/7/68 (Example 1-1) or with the mass ratio of 1.5/8.9/89.6 (Example 1-2) using a stirrer (equipment used: model STIRRER SSR, IWAKI, rotation speed: 400 to 600 rpm, temperature: 25±5° C.) so as to prepare the formation material of the adhesive layer.

Subsequently, a slide glass having a thickness of 1.3 mm was set as a spacer on each of the four sides on a glass plate, the above prepared solution was poured onto the glass plate, and another glass plate was covered from above to sandwich the glass plate. It was then frozen at −20° C. and cut into a rectangular shape having a length of 11 mm×a width of 5 mm in the frozen state. These defrosted at 5° C. was used as the water-containing base adhesive skin patch with a water-soluble steroid.

### Example 2: Water-Containing Base Adhesive Skin Patch with Liposoluble Steroid

In the formation material of the adhesive layer, the water-containing adhesive skin patch of Example 2 was obtained in the same manner with Example 1 except that clobetasol propionate (CP), which is a liposoluble steroid, was used in place of the water-soluble steroid, and that CP/PVA/water=1.5/8.9/89.6 (mass ratio).

### Test Example 1: Drug Skin Permeation Test (1) by Administration of Water-Containing Base Adhesive Skin Patch after Microneedle Perforation, by Using Skin Resected from Hairless Mouse

### <Test Method>

Perforation of microneedle was performed at the perforation rate of 8.5 m/s onto the abdominal resected skin of a hairless mouse (male, 7 weeks old, Nippon SLC) placed on a cork board, by using a microneedle array shown below.

The water-containing base adhesive skin patch of Example 1 (Example 1-1: DSP/PVA/water=25/7/68 (by mass)) or Example 2 is placed on the skin portion to be perforated, and a cathereep (polyurethane film, Nichiban Co., Ltd.) is placed thereon as an adhesive tape for fixation. Then the skin surface is installed into a vertical diffusion cell for skin penetration test (inner diameter: 20 mmφ, receiver capacity: about 16 mL, effective diffusion area: 3.14 cm²).

Warm water at 32° C. was passed through the jacket of the diffusion cell, and the following receiver liquid was added into a receiver chamber to start the skin permeation test. At every elapse of a fixed time from the start of the test, each 0.5 mL of the receiver liquid was sampled from the sampling port of the diffusion cell, and the same amount of receiver liquid was replenished. Then, the same amount of methanol was added to the collected receiver solution and subjected to centrifugation so as to collect supernatant as a sample. The permeated drug amount of the sample obtained was quantified by HPLC, and the cumulative permeation amount thereof was calculated.

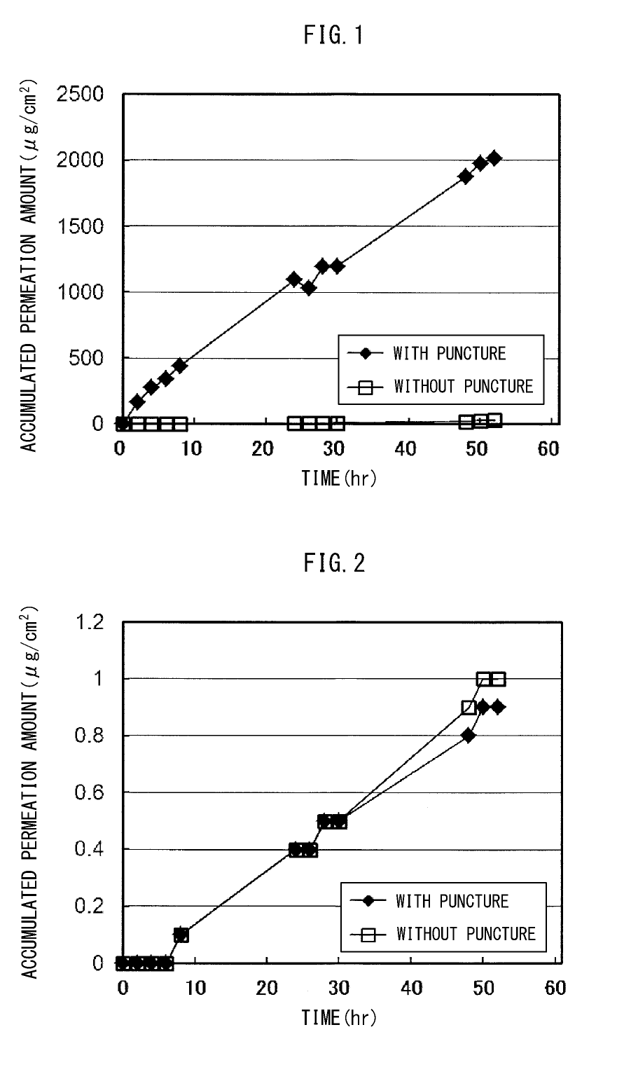

In addition to the above, as a comparative example, a skin permeation test using the water-containing base adhesive skin patch of Example 1 (Example 1-1: DSP/PVA/water=25/7/68 (mass ratio)) and Example 2 was performed in the same manner except that the microneedle treatment was not performed (no perforation). Results that have been obtained are shown in Table 1 (with perforation treatment), Table 2 (with no perforation treatment), FIG. 1 (application of water-containing base adhesive skin patch with water-soluble steroid) and FIG. 2 (application of water-containing base adhesive skin patch with liposoluble steroid).

“Microneedle Array”

A microneedle array (rectangular shape with length of 11 mm×width of 5 mm) having 305 conical microneedles per 1 array (height 300 μm×bottom diameter 300 μm) made of polycarbonate was used.

“Receiver Liquid”

### Example 1 (Example 1-1) (Water-Containing Base Adhesive Skin Patch with Water-Soluble Steroid): Phosphate Buffer Solution (pH: 7.4)

### Example 2 (Water-Containing Base Adhesive Skin Patch with Liposoluble Steroid): 20% Polyethylene Glycol Solution (PEG Molecular Weight, Etc.: 380-420, Manufactured by Kanto Chemical Co.)

“HPLC”

### Example 1 (Example 1-1) (Water-Containing Base Adhesive Skin Patch with Water-Soluble Steroid)

Apparatus: LC-2010HT (manufactured by Shimadzu Corporation)

Column: Kinetex C8 100 A, 5 μm, 4.6×250 mm (Phenomenex)

Column temperature: 40° C.

Injection volume: 50 μL

Flow rate: 0.65 mL/min

Detection wavelength: 220 nm

Mobile phase: 0.1% phosphoric acid solution/acetonitrile/methanol=54/35/11

### Example 2 (Water-Containing Base Adhesive Skin Patch with Liposoluble Steroid)

Apparatus: LC-2010HT (manufactured by Shimadzu Corporation)

Column: Mightysil RP-18 GP, 5 μm, 4.6×150 mm (Kanto Kagaku Co., Ltd.)

Column temperature: 25° C.

Injection volume: 30 μL

Flow rate: 1.04 mL/min

Detection wavelength: 240 nm

Mobile phase A: 0.05 M PBS/acetonitrile/methanol=35/45/20

Mobile phase B: Methanol

TABLE 1
Drug cumulative permeation amount (with
microneedle perforation) (N = 3)

| | | Water-containing base | | Water-containing base | |
|---|---|---|---|---|---|
| | | adhesive skin patch with | | adhesive skin patch with | |
| | | water-soluble steroid | | liposoluble steroid | |
| | | (Example 1-1) | | (Example 2) | |
| | Time | Average | Standard | Average | Standard |
| | (hr) | value | error | value | error |
| Cumulative | 0 | 0.0 | 0.0 | 0.0 | 0.0 |
| permeation | 2 | 163.7 | 24.0 | 0.0 | 0.0 |
| amount | 4 | 274.2 | 21.0 | 0.0 | 0.0 |
| (μg/cm²) | 6 | 343.1 | 44.1 | 0.0 | 0.0 |
| | 8 | 439.8 | 61.6 | 0.1 | 0.0 |
| | 24 | 1095.2 | 106.8 | 0.4 | 0.0 |
| | 26 | 1029.2 | 119.8 | 0.4 | 0.0 |
| | 28 | 1194.5 | 142.8 | 0.5 | 0.0 |
| | 30 | 1197.4 | 209.7 | 0.5 | 0.0 |
| | 48 | 1877.7 | 206.0 | 0.8 | 0.0 |
| | 50 | 1976.1 | 262.1 | 0.9 | 0.0 |
| | 52 | 2017.6 | 263.6 | 0.9 | 0.0 |

TABLE 2
Drug cumulative permeation amount (without
microneedle perforation) (N = 3)

| | | Water-containing base | | Water-containing base | |
|---|---|---|---|---|---|
| | | adhesive skin patch with | | adhesive skin patch with | |
| | | water-soluble steroid | | liposoluble steroid | |
| | | (Example 1-1) | | (Example 2) | |
| | Time | Average | Standard | Average | Standard |
| | (hr) | value | error | value | error |
| Cumulative | 0 | 0.0 | 0.0 | 0.0 | 0.0 |
| permeation | 2 | 0.5 | 0.0 | 0.0 | 0.0 |
| amount | 4 | 0.7 | 0.1 | 0.0 | 0.0 |
| (μg/cm²) | 6 | 0.9 | 0.1 | 0.0 | 0.0 |
| | 8 | 1.0 | 0.0 | 0.1 | 0.1 |
| | 24 | 4.5 | 0.7 | 0.4 | 0.1 |
| | 26 | 4.9 | 0.9 | 0.4 | 0.1 |
| | 28 | 5.8 | 1.2 | 0.5 | 0.1 |
| | 30 | 7.1 | 1.8 | 0.5 | 0.1 |
| | 48 | 22.5 | 10.2 | 0.9 | 0.1 |
| | 50 | 27.3 | 13.3 | 1.0 | 0.1 |
| | 52 | 30.7 | 15.4 | 1.0 | 0.1 |

As shown in Tables 1 and 2, in the water-containing base adhesive skin patch with water-soluble steroid of Example 1 (Example 1-1), by conducting the microneedle perforation treatment on skin the adhesive skin patch is to be applied (Table 1), the cumulative skin permeation amount was remarkably increased as compared with the case without the microneedle perforation treatment (Table 2). Specifically, the cumulative skin permeation amount after 24 hours of application was 4.5 μg/cm² in the case of not performing the microneedle perforation treatment, and 1095.2 μg/cm² in the case of performing the microneedle perforation treatment. The cumulative skin permeation amount was about 243 times higher (see FIG. 1).

On the other hand, in the water-containing base adhesive skin patch with liposoluble steroid of Example 2, regardless of whether to perform the microneedle perforation treatment, compared with the water-containing adhesive skin patch with the water-soluble steroid of Example 1 (Example 1-1), the cumulative skin permeation amount was extremely low. In addition, no increase in the cumulative skin permeation amount due to the microneedle perforation treatment could be observed (see FIG. 2).

### Test Example 2: Drug Skin Permeation Test (2) by Administration of Water-Containing Base Adhesive Skin Patch after Microneedle Perforation, by Using Skin Resected from Hairless Mouse

<Test Method>

Perforation of microneedle was performed at the perforation rate of 6.0 m/s onto the abdominal resected skin of a hairless mouse (male, 7 weeks old, Nippon SLC) placed on a cork board, by using a microneedle array shown below.

The water-containing base adhesive skin patch of Example 1 (Example 1-2: DSP/PVA/water=1.5/8.9/89.6 (by mass)) or Example 2 is placed on the skin portion to be perforated, and a cathereep (polyurethane film, Nichiban Co., Ltd.) is placed thereon as an adhesive tape for fixation. Then the skin surface is installed into a vertical diffusion cell for skin penetration test (inner diameter: 20 mmφ, receiver capacity: about 16 mL, effective diffusion area: 3.14 cm²).

Warm water at 32° C. was passed through the jacket of the diffusion cell, and the following receiver liquid was added into a receiver chamber to start the skin permeation test. At every elapse of a fixed time from the start of the test, each 0.5 mL of the receiver liquid was sampled from the sampling port of the diffusion cell, and the same amount of receiver liquid was replenished. Then, the same amount of methanol was added to the collected receiver liquid and subjected to centrifugation so as to collect supernatant as a sample. The permeated drug amount of the sample obtained was quantified by HPLC, and the cumulative permeation amount thereof was calculated.

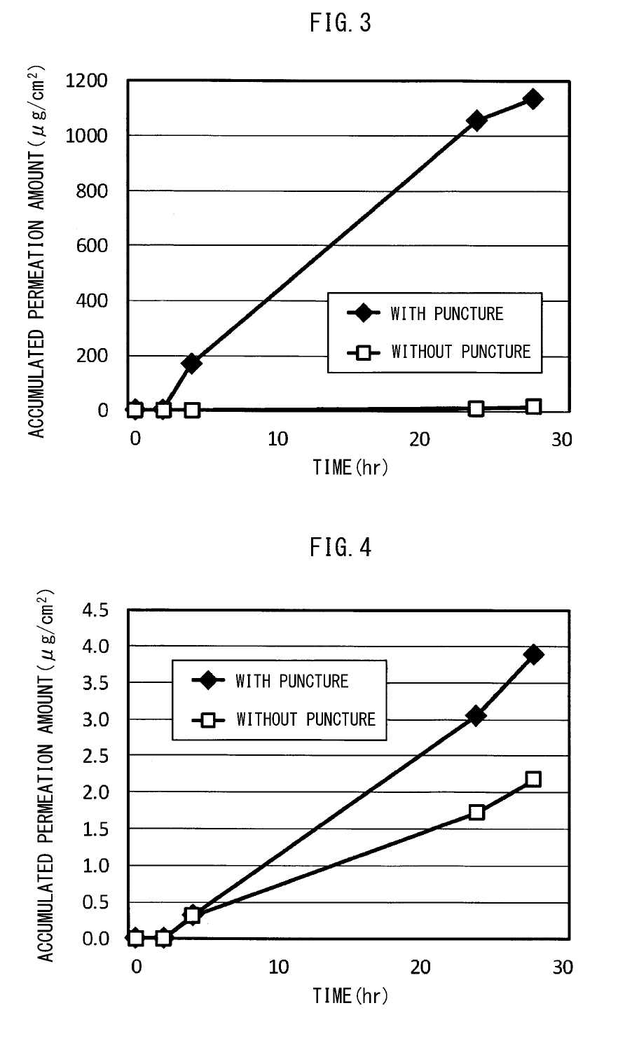

In addition to the above, as a comparative example, a skin permeation test using the water-containing base adhesive skin patch of Example 1 (Example 1-2: DSP/PVA/water=1.5/8.9/89.6 (mass ratio)) and Example 2 was performed in the same manner except that the microneedle treatment was not performed (no perforation). Results that have been obtained are shown in Table 3 (with perforation treatment), Table 4 (with no perforation treatment), FIG. 3 (application of water-containing base adhesive skin patch with water-soluble steroid) and FIG. 4 (application of water-containing base adhesive skin patch with liposoluble steroid).

<Microneedle Array>

A circular microneedle array (diameter 0.8 cm) having 305 conical microneedles per 1 array (height 300 μm×bottom diameter 300 μm) made of polycarbonate was used.

### Receiver Liquid

### Example 1 (Example 1-2) (Water-Containing Base Adhesive Skin Patch with Water-Soluble Steroid): Phosphate Buffer Solution (pH: 7.4)

### Example 2 (Water-Containing Base Adhesive Skin Patch with Liposoluble Steroid): 20% Polyethylene Glycol Solution (PEG Molecular Weight, Etc.: 380-420, Manufactured by Kanto Chemical Co.)

### HPLC

### Example 1 (Example 1-2) (Water-Containing Base Adhesive Skin Patch with Water-Soluble Steroid)

Apparatus: LC-2010HT (manufactured by Shimadzu Corporation)

Column: Kinetex C8 100 A, 5 μm, 4.6×250 mm (Phenomenex)

Column temperature: 40° C.

Injection volume: 50 μL

Flow rate: 0.65 mL/min

Detection wavelength: 254 nm

Mobile phase: 0.1% phosphoric acid solution/acetonitrile/methanol=54/35/11

### Example 2 (Water-Containing Base Adhesive Skin Patch with Liposoluble Steroid)

Apparatus: LC-2010HT (manufactured by Shimadzu Corporation)

Column: Mightysil RP-18 GP, 5 μm, 4.6×150 mm (Kanto Kagaku Co., Ltd.)

Column temperature: 25° C.

Injection volume: 30 μL

Flow rate: 1.04 mL/min

Detection wavelength: 240 nm

Mobile phase A: 0.05 M PBS/acetonitrile/methanol=35/45/20

Mobile phase B: Methanol

TABLE 3
Drug cumulative permeation amount (with
microneedle perforation) (N = 3)

| | | Water-containing base | | Water-containing base | |
|---|---|---|---|---|---|
| | | adhesive skin patch with | | adhesive skin patch with | |
| | | water-soluble steroid | | liposoluble steroid | |
| | | (Example 1-2) | | (Example 2) | |
| | Time | Average | Standard | Average | Standard |
| | (hr) | value | error | value | error |
| Cumulative | 2 | 0.0 | 0.0 | 0.0 | 0.0 |
| permeation | 4 | 170.2 | 45.3 | 0.3 | 0.2 |
| amount | 24 | 1054.0 | 88.7 | 3.1 | 0.6 |
| (μg/cm²) | 28 | 1136.4 | 88.8 | 3.9 | 0.8 |

TABLE 4
Drug cumulative permeation amount (without
microneedle perforation) (N = 3)

| | | Water-containing base | | Water-containing base | |
|---|---|---|---|---|---|
| | | adhesive skin patch with | | adhesive skin patch with | |
| | | water-soluble steroid | | liposoluble steroid | |
| | | (Example 1-2) | | (Example 2) | |
| | Time | Average | Standard | Average | Standard |
| | (hr) | value | error | value | error |
| Cumulative | 2 | 0.0 | 0.0 | 0.0 | 0.0 |
| permeation | 4 | 0.2 | 0.1 | 0.3 | 0.2 |
| amount | 24 | 8.1 | 2.6 | 1.7 | 0.2 |
| (μg/cm²) | 28 | 15.1 | 6.6 | 2.2 | 0.2 |

As shown in Tables 3 and 4, in the water-containing base adhesive skin patch with water-soluble steroid of Example 1 (Example 1-2), by conducting the microneedle perforation treatment on the skin the adhesive skin patch was applied (Table 3), a cumulative skin permeation amount was remarkably increased as compared with the case without the microneedle perforation treatment (Table 4). Specifically, the cumulative skin permeation amount after 24 hours of application was 8.1 μg/cm² in the case of not performing the microneedle perforation treatment, and 1054.0 μg/cm² in the case of performing the microneedle perforation treatment. The cumulative skin permeation amount was about 130 times higher (see FIG. 3).

On the other hand, in the water-containing base adhesive skin patch with liposoluble steroid of Example 2, regardless of whether to perform the microneedle perforation treatment, compared with the water-containing adhesive skin patch with the water-soluble steroid of Example 1 (Example 1-2), the cumulative skin permeation amount was extremely low. In addition, no increase in the cumulative skin permeation amount due to the microneedle perforation treatment could be hardly observed compared with the water-containing adhesive skin patch with water-soluble steroid (see FIG. 4)

As shown in the results of these test examples, even if the water-soluble steroid (DSP) and the liposoluble steroid (CP) are set to have the same concentration in the water-containing adhesive skin patch, the results of the same tendency with the previous Test Example 1 were obtained.

### Test Example 3: Measurement Test of Drug Concentration in Tissue Near Meibomian Glands by Administration of Water-Containing Adhesive Skin Patch after Microneedle Perforation, by Using Rabbit Eyelid Skin

<Test Method>

Anesthesia was given to rabbits (Slc: JW/CSK, Japanese white species, Japan SLC, Inc.) by inhalation of isoflurane, and the periphery of the upper eyelid was shaved using hair clippers and a shaver until skin was exposed. The skin of the upper eyelid was fixed with entropion forceps, and the microneedle was then perforated on the shaved skin of the upper eyelid with a microneedle array as shown below at the perforation rate of 6.0 m/s.

The water-containing adhesive skin patch with water-soluble steroid (provided that the size is 1.5 cm×0 8 cm, and the application area is 1.2 cm²) of Example 1 (Example 1-1: DSP/PVA/water=25/7/68 (mass ratio)) was applied to the skin of the perforated portion for 16 hours [a cathereep (polyurethane film, Nichiban Co., Ltd.) was placed thereon as an adhesive tape for fixation]. The patch was then released. After cleaning the portion that the adhesive skin patch has been applied with gauze, the surrounding tissues including meibomian glands (hereinafter referred to as meibomian gland surrounding tissues) were excised from the applied portion when the patch was just released (that is, after 16 hours after application), and respectively after 24 hours, 72 hours, 168 hours or 336 hours of application. In order to extract drug in each tissue, the excised meibomian gland surrounding tissues were immersed in water/acetonitrile/methanol=54/35/11 for 12 to 24 hours and measured for the drug concentration therein using the measuring method of HPLC analysis described in detail below.

<Microneedle Array>

A circular microneedle array (diameter 0.8 cm) having 305 conical microneedles (height 300 μm×bottom diameter 300 μm) made of polycarbonate was used.

<Measurement Method of Drug Concentration in Meibomian Gland Surrounding Tissues>

The meibomian gland surrounding tissues that have been excised were cut with scissors and sent to a centrifuge tube. Solution of 1 mL in water/acetonitrile/methanol=54/35/11 was added into the tube and left to stand still in a refrigerator for 12 to 24 hours. The tube was then subjected to centrifugation at 10,000 rpm for 10 minutes with a centrifuge, and 0.8 mL of the supernatant thereof was transferred to another test tube. The solvent was eliminated and dried through nitrogen-gas blowing, and 0.5 mL of solution in water/acetonitrile/methanol=54/35/11 was added and redissolved. The test tube was then centrifuged at 10,000 rpm for 10 minutes using a centrifuge. 0.4 mL of the supernatant thereof was filtered, and the concentration thereof was measured by the HPLC analysis.

<HPLC>

Apparatus: LC-2010HT (manufactured by Shimadzu Corporation)

Column: Kinetex 5 C8 100 A, 5 μm, 4.6×250 mm (Shimadzu GLC Co., Ltd.)

Column temperature: 40° C.

Injection volume: 50 μL

Flow rate: 0.65 mL/min

Detection wavelength: 254 nm

Mobile phase: 0.1% phosphate buffer solution/acetonitrile/methanol=54/35/11

TABLE 5
Drug concentration in meibomian gland surrounding tissues
(N = 3 but 24 hr and 336 hr is N = 2)

| | |
|---|---|
| Time* | Drug concentration (μg/g) |

| (hr) | Average value | Standard error |
|---|---|---|
| Immediately | 8.2 | 6.4 |
| after release | | |
| (16)** | | |
| 24 | 5.2 | 3.1 |
| 72 | 1.3 | 1.7 |
| 168 | 0.1 | 0.2 |
| 336 | 0.5 | 0.7 |

*Time indicates an elapsed time after application (that is, administration)
**Adhesive skin patch is released from applied portion after 16 hr from application

When a hydrophobic steroid is administered through the application of ointment onto the upper eyelid skin, it has been known that the intra-tissue concentration (Cmax) of about 2.1 μg/g is observed in palpebral conjunctiva including meibomian glands after 15 minutes from the administration (Pharmacology Review(s) 2010; NDA 200-738).

In the present invention, by administering the water-soluble steroid in the form of the adhesive skin patch after the microneedle perforation treatment has been conducted, it has been acknowledged that the drug concentration in the meibomian gland surrounding tissues was about 3.9 times higher than that of the ointment.

In addition, according to the present invention, the drug concentration in the meibomian gland surrounding tissues can increase up to 8.2 μg/g in 16 hours after administration, meaning that more than 0.050 μg/g of a sufficient amount of steroid will be deliverable to diseased portions for a short period of time. Further, it is possible to retain more than 0.050 μg/g of the sufficient amount of steroid in the diseased portions for 336 hours after administration, that is, for 2 weeks.

In the present invention as discussed hereinabove, compared with conventional treatment, it is possible to administer a sufficient amount of dosing to diseased portions of the meibomian gland surrounding tissues for a short period of time and to retain the concentration of the drug at the diseased portions. This can avoid the long-term use of steroid. For this reason, it has been known that users do not experience any of intraocular pressure elevation, cataract, corneal epithelial disorder, and wound healing delay, corticosteroid uveitis, mydriasis, ptosis and infection in which to be induced by long-term use of ophthalmic steroids such as steroid eye-wash, ophthalmic ointment and the like, as well as other serious side-effects such as transient ocular discomfort and steroid-induced calcium deposits. Accordingly, the present invention is considered to be a therapeutic method with less burden on patients.

1. A transdermal drug delivery system for administering a drug for treating ophthalmic diseases via an eyelid skin that has been treated by a microneedle array, wherein
the drug is a water-soluble steroid, and
the transdermal drug delivery system is a water-containing base adhesive skin patch.

2. The transdermal drug delivery system accordingly to claim 1, wherein
the transdermal drug delivery system is a water-containing adhesive skin patch comprising either adhesive hydrogel, or non-adhesive hydrogel and an adhesive tape for fixation, and
the hydrogel contains a water-soluble steroid as the drug.

3. The transdermal drug delivery system according to claim 1, wherein
the transdermal drug delivery system is a water-containing base adhesive skin patch obtained by which a water-containing base adhesive layer is arranged on a backing film, and
the water-containing base adhesive layer contains a water-soluble steroid as the drug.

4. The transdermal drug delivery system according to claim 1, wherein the drug is at least one of the water-soluble steroids selectable from a range of −5 to 0 in octanol/water distribution coefficient (log D).

5. The transdermal drug delivery system according to claim 4, wherein
the drug is at least one of the water-soluble steroids selectable from the group consisting of dexamethasone sodium phosphate, dexamethasone metasulfobenzoate sodium, hydrocortisone sodium phosphate, hydrocortisone sodium succinate, prednisolone sodium phosphate, prednisolone sodium succinate, methylprednisolone sodium succinate, and betamethasone sodium phosphate.

6. The transdermal drug delivery system according to claim 2, wherein the water-containing base adhesive layer or hydrogel contains polyvinyl alcohol.

7. The transdermal drug delivery system according to claim 2, wherein the water-containing base adhesive layer or hydrogel contains at least one kind selectable from the group consisting of polyacrylic acid and salt thereof.

8. The transdermal drug delivery system according to claim 1, wherein the ophthalmic disease is at least one disease selectable from the group consisting of chalazion, blepharitis, allergic conjunctivitis, vernal keratoconjunctivitis and meibomian gland dysfunction.

9. A method for treating ophthalmic diseases, the method comprising:
a process of perforating a microneedle on an eyelid skin surface by using a microneedle array; and
a process of locally applying a transdermal drug delivery system according to claim 1 to a portion of an eyelid skin on which perforation of the microneedle has been performed.

10. A set for treating ophthalmic diseases, wherein the set includes a microneedle array for perforating an eyelid skin, a support base for eyelid skin, and a transdermal drug delivery system according to claim 1.

11. The set of treating ophthalmic diseases according to claim 10, wherein the support base for eyelid skin is either entropion forceps or a cornea protection plate (lid plate).

12. A transdermal drug delivery system for administering a drug for treating ophthalmic diseases via an eyelid skin that has been treated by a microneedle array, wherein
when the drug is a liposoluble steroid, the transdermal drug delivery system further comprises a water-soluble additive, the additive being produced by making the liposoluble steroid esterified to become water soluble, and
the transdermal drug delivery system is a water-containing base adhesive skin patch.

13. The transdermal drug delivery system according to claim 3, wherein the water-containing base adhesive layer or hydrogel contains polyvinyl alcohol.

14. The transdermal drug delivery system according to claim 3, wherein the water-containing base adhesive layer or hydrogel contains at least one kind selectable from the group consisting of polyacrylic acid and salt thereof.
